(19) United States
(12) Patent Application Publication
(10) Pub. No.: US 2005/0192495 A1
(43) Pub. Date: Sep. 1, 2005

(54) Medical examination apparatus having means for performing correction of settings

(76) Inventors: Sherif Makram-Ebeid, Eindhoven (NL); Pierre LeLong, Nogent Sur Marne (FR); Bert Leo Alfons Verdonck, Eindhoven (NL); Jean-Pierre Franciscus Alexander Maria Ermes, Eindhoven (NL)

Correspondence Address: PHILIPS INTELLECTUAL PROPERTY & STANDARDS, 595 MINER ROAD, CLEVELAND, OH, 44143, US

(21) Appl. No.: 10/499,310

(22) PCT Filed: Dec. 16, 2002
(86) PCT No.: PCT/IB02/05493

§ 371 Date: Jun. 18, 2004

(30) Foreign Application Priority Data
Dec. 28, 2001 (EP) 01403382.3

(51) Int. Cl. G06K 9/00; A61B 5/05

(52) U.S. Cl. 600/407; 382/325

(57) ABSTRACT

A medical examination apparatus including an imaging means (2,3), a viewing system (4), wherein image data processing means (5) is arranged to facilitate production of different images of a feature of interest such that the pose of the feature is comparable in the different images. The image data processing means (5) estimates the pose of the anatomical feature in a first image generated by the imaging means, produces imaging means control data indicative of the desired imaging geometry for controlling one or more parameters of the imaging means (2,3) for producing a further image having the feature of interest in the estimated pose, and outputs the produced imaging means control data. The output control data may be output in a viewable form and/or output directly to the imaging means (2,3) so as automatically to control the parameters thereof. The output control data can also control the imaging means so as to produce an image having desired intensity characteristics.

### FIELD OF THE INVENTION

The present invention relates to a medical examination apparatus having means for performing correction of settings in order to facilitate comparison of medical images, as well as to a medical viewing system, and a computer program product.

### BACKGROUND OF THE INVENTION

With the widespread adoption of medical imaging technology, such as x-ray imaging apparatus, CT scanners and the like, there has been a need for improved medical viewing systems enabling the image data to be visualized in a form that is useful to medical practitioners. Most medical examination apparatus are associated with a viewing system, which comprises some computer-based data processing equipment capable of processing the image data and generating a viewable representation of an imaged element, for example a body part, organ, etc., in real-time. In general, it is desirable for such a viewing system to be interactive, enabling the medical practitioner to influence the image that is acquired and/or the representation of the image data. A workstation remote from the imaging apparatus is also often used for post-processing of the acquired image data.

One medical viewing system designed to facilitate analysis of the movement of artificial joints is described in the article “An interactive system for kinematic analysis of artificial joint implants” by Sarojak et al, Proc. of the 36^{th} Rocky Mountain Bioengineering Symposium, 1999. The aim of this system is to be able to generate images of a total joint arthroplasty (TJA) implant in different positions, so as to be able to study the nature of the motions involved when the joint is in function. In order to facilitate the analysis of joint motion, this system processes image data for each position of the joint, in order to be able to quantify the “pose” of the implant in the image in question. The “pose” is measured with reference to a computer aided design model of the implant. By estimation of the “pose” of the implant, it is to be understood the estimation of the viewing angles of the implant from a single two-dimensional (2D) projection or 2D image of the implant, knowing the three-dimensional (3D) shape of the implant. This publication describes the use of a pattern matching technique for estimating the pose of the implant, i.e. the angle of the implant relative to the 2D image frame coordinates system. The work of the authors was aimed at making kinetic studies of the implant in the knee of the patient. This publication does not disclose attempts to make long term comparisons between follow-up images. Neither did they try to apply the technique to another more complex implant such as a hip prosthesis.

It is often desirable to be able to compare medical images of the same anatomical feature acquired at different times, typically so as to detect medically significant changes. For example, in the field of orthopedic surgery, when a prosthesis, such as a replacement hip, is implanted, the prosthesis can cause changes in the surrounding structures. Moreover, the position of the prosthesis can change over time and the prosthesis can be subject to wear. In order to monitor such developments, it is desirable to generate an image of the prosthesis and its environment right after the operation implanting the prosthesis, and to generate follow-up images at intervals afterwards, such as after one week, then one month, etc., right up to several years later. By comparison of the images taken at different times, the medical practitioner can assess how the prosthesis is affecting its environment, and whether the prosthesis is moving and/or subject to wear.

When using a current medical examination apparatus with an associated viewing system, it is not a simple matter to compare medical images of the same anatomical feature taken at different times. The position of the anatomical feature relative to the medical examination apparatus is not necessarily constant between images, causing differences in the geometry of the feature in the image. Furthermore, the images to be compared may be taken using different imaging devices and/or the settings of the imaging device of the medical examination apparatus may be different between the images, causing differences in the relative intensities of pixels in the image as well as in the geometry of the feature. As indicated, these differences in the imaging conditions affect the images to be compared. Thus, when viewing the images to be compared, it becomes difficult for the medical practitioner to differentiate between true changes in the anatomical feature and its environment and apparent changes in the image that are due merely to differences in the imaging conditions.

By the way, it is to be understood that in this document the expression “feature of interest” is used broadly to designate any feature or region in the body (whether human or animal), whether a bone, a vessel, an organ, a fluid, or anything else, and includes artificial elements implanted into or attached to the body.

### SUMMARY OF THE INVENTION

It is an object of the present invention to provide a medical examination apparatus associated to a viewing system having means to permit a practitioner to perform accurate comparisons of a same feature of interest acquired in different medical images, for instance generated at different times, at different stages of the follow-up procedure.

More particularly, it is an object of the present invention to provide such a viewing system having means to estimate the pose of a prosthesis in a current view, assuming that the pose of a 3D model of the prosthesis is previously known. This should allow the practitioner to make sure that the prosthesis will be seen with substantially the same viewing angles in one follow-up image or in a series of follow-up images. In case of differences with the previously known viewing angles, information is provided to the practitioner relative to settings of the medical examination apparatus using the viewing system. The medical examination apparatus settings are logged and the information of the corresponding data is displayed on a part of the display system of the viewing system in relation with the image to be corrected. These correction data will permit the practitioner of correcting the settings of the medical examination apparatus related to the “pose”, using the viewing system, hence will permit of reducing, in medical images of the same feature of interest generated at different times, discrepancies arising from differences in imaging conditions.

In a first embodiment, the system of the invention, has means to facilitate comparison of medical images of a feature of interest taken at different times, comprising first means for indicating corrections to be applied to the “pose”, i.e. the viewing angles, of the feature of interest represented in a follow-up image with respect to the pose of the same feature of interest represented in an image of reference. According to the present invention, in order to avoid gross differences in pose, an initial image, called image of reference, representing the feature of interest is analyzed in order to estimate the pose of said feature, called reference pose. The reference pose may be estimated relative to the feature of interest in a reference 3D model or to a discriminating part of the feature of interest in a corresponding reference 2D image. Then, when a follow-up image is further formed, control data is emitted which instructs the operator of the imaging apparatus how to modify the imaging apparatus settings and/or to arrange the patient in order to produce said follow-up image in which the feature of interest has a pose that is the same or substantially close to that of the same feature of interest in the reference image. An advantage is that, in the follow-up image, the feature of interest is seen with substantially the same viewing angles and magnification as in the reference image.

In an other embodiment, the system of the invention has second means to facilitate comparison of medical images of a feature of interest taken at different times, comprising second means for indicating corrections of the settings of the exposition light generator used to acquire a follow-up image with respect to the settings of the exposition light generator used to acquire the reference image. According to the invention, an image intensity calibration phantom is added in order to fully characterize the exposition light generator settings, for example x-ray generator settings, for correcting intensity level discrepancies between the follow-up image and the reference image. Then, when a follow-up image is further formed, control data are emitted using the viewing system, which instruct the operator of the medical examination apparatus how to arrange the settings of the medical examination apparatus in order to produce said follow-up image in which the feature of interest has gray levels substantially close to that of the same feature in the reference image. An advantage is that the follow-up image and the image of reference can be compared with accuracy.

This control data may be generated based on a comparison of a feature of interest of a reference image with the same feature of an image produced in a “trial run” just prior the acquisition of a final follow-up image. The trial image is an intermediate image between the reference image and the corrected final image, on which differences between the pose of the feature of interest and its gray levels are estimated with respect to the reference image, from which control data are emitted. In such a case, the control data may be associated to instructions to the operator as to how to change the set-up of the medical examination apparatus and/or the position of the patient so as to obtain a final follow-up image having the feature of interest in the desired pose. In other words, the output control data may be indicative of desired values of one or more of geometrical parameters of the medical examination apparatus; and/or the output control data may be indicative of changes to be made to such parameters of the medical examination apparatus, so as to obtain the final follow-up image having the feature of interest in the desired pose. The control data may additionally instruct the operator how to adjust setting parameters of the medical examination apparatus related to the intensity levels of the image, such as the exposition light generator settings of voltage and current intensity of said exposition light generator.

### BRIEF DESCRIPTION OF THE DRAWINGS

The invention is described in detail in reference to the following schematic drawings:

FIG. 1A is a diagram illustrating the main components of a medical examination apparatus; FIG. 1B represents the orthogonal 3D referential of the geometrical settings of the medical examination apparatus; and FIG. 1C represents the referential of a 2D image frame;

FIG. 2 is a flow diagram illustrating major steps in the operation of the medical examination apparatus of FIG. 1;

FIG. 3 is an example of a reference image, in which FIG. 3A shows an x-ray 2D projection image of a hip prosthesis, and FIG. 3B shows the outline of a discriminating portion DP1 of the hip prosthesis; and FIG. 3C shows a gray level calibration phantom to be associated to the taking of the projection images;

FIG. 4 is an example of a trial image to be compared with the reference image of FIG. 3, in which FIG. 4A shows an x-ray 2D projection image of the same hip prosthesis, FIG. 4B shows the outline of a discriminating portion DP2 of the hip prosthesis to be compared with the outline of the same discriminating portion DP1 of FIG. 3B; and FIG. 4C shows a comparison of the calibration phantom gray levels in the reference Image (Image I1) and trial Image (Image I2).

### DESCRIPTION OF EMBODIMENTS

The present invention will be described in detail below with reference to embodiments in which x-ray examination apparatus and an associated viewing system are used to produce and process images of a hip prosthesis regarded as a feature of interest. However, it is to be understood that the present invention is applicable more generally to medical examination apparatus and to viewing systems using other types of imaging technology. There is substantially no limit on the human or animal feature of interest that can be the object of the images to be processed according to the invention.

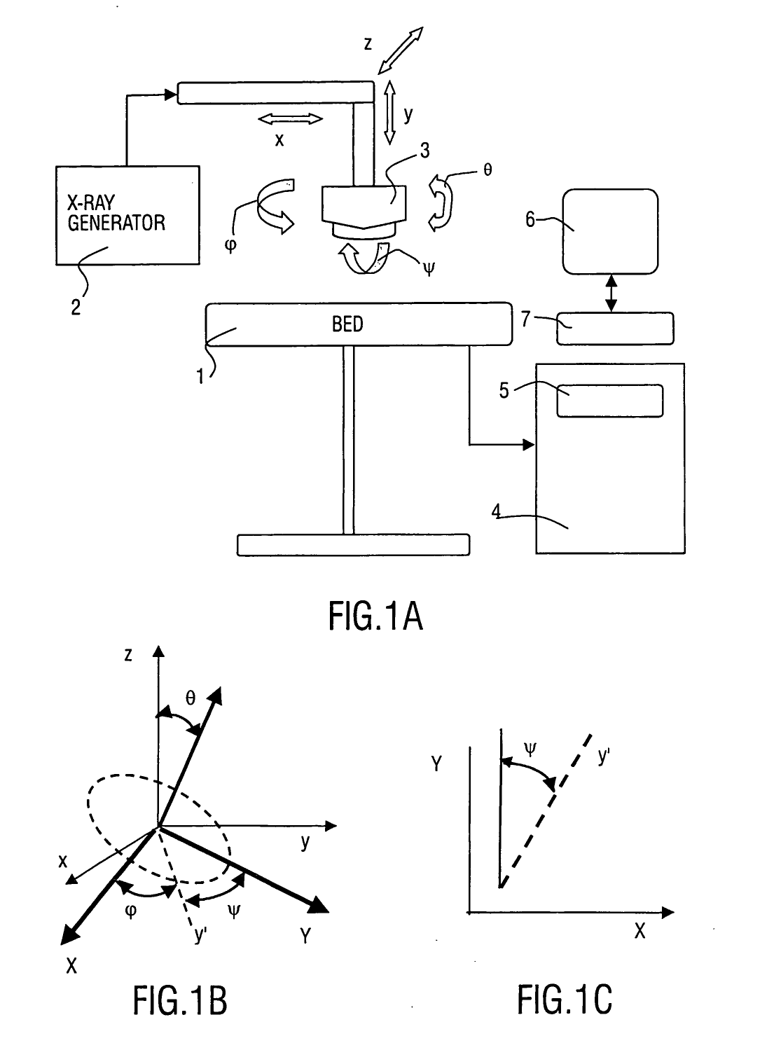

FIG. 1A is a diagram showing the main components of a medical examination apparatus and a viewing system. The medical examination apparatus includes a bed 1 upon which the patient will lie, an x-ray generator 2 associated with x-ray imaging device 3 for producing an image of an anatomical feature of interest of the patient. The medical examination apparatus comprises or is associated to a viewing system including a computer system 4 for processing the image data produced by the x-ray imaging device 3. The viewing system is optimized to enable different images of the feature of interest to be produced such that the pose of the feature of interest is comparable in the different images. Typically, the different images will be generated at different times and a medical practitioner will wish to compare the images so as to identify developments occurring in the patient's body during the interval intervening between the taking of the different images.

The patient may be presented to the x-ray examination apparatus on a support other than a bed, or may stand so as to present the whole or a part of himself in a known positional relationship relative to the examination apparatus, in a well-known manner. Similarly, known x-ray examination apparatus may be used. In the viewing system, the computer system 4 includes data processing means 5, a display screen 6 and a keyboard 7 for entry of data and/or instructions. The viewing system may also include or be connected to other conventional elements and peripherals, as is generally known in this field. For example, the viewing system may be connected by a bus to local or remote work stations, printers, archive storage, etc.

In this invention, 2D x-ray images of the prosthesis and its surrounding region are assumed to be available in digital format. Furthermore, a 3D model of the prosthesis is also assumed to be available. It is possible to acquire a 3D reference image of the prosthesis through 3D imaging of similar prosthesis of the same make or by using a 3D CAD model provided by the implant manufacturer. Another possibility for acquiring a 3D reference image of the prosthesis is to perform a 3D imaging of the patient right after the operation for the region around the prosthesis.

The viewing system of the invention has data processing means 5 to perform the procedure hereafter described.

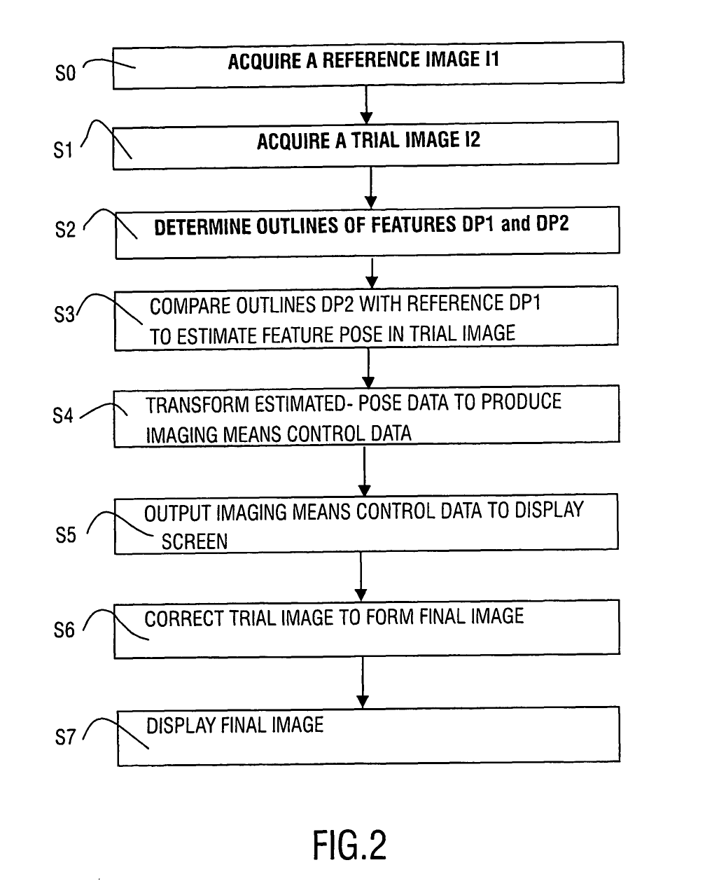

FIG. 2 is a flow diagram of the functions performed by the data processing means 5 of the medical examination apparatus and viewing system of FIG. 1A. Preferably, before the image data processing means described below are applied to images produced by the x-ray imaging device 3 of FIG. 1A, standard x-ray image calibration and correction procedures are applied to the images. Such procedures include, for example, corrections for pincushion and earth magnetic field distortions, and for image intensifier vignetting effects.

As shown in FIG. 2, the viewing system uses the acquisition means 3 to perform an acquisition step S1 of acquiring a “trial” image I2 of the feature of interest, in a given patient. The trial image will be an intermediate image that is analyzed to determine geometrical data settings and light intensity settings in order to finally obtain a follow-up image with substantially the same viewing angles and geometrical parameters called “pose” and substantially the same light exposition conditions as the reference image. Typically, this trial image will be acquired by using the x-ray imaging device 3 to produce a “test shot” of the appropriate region of the patient's body, for example the hip region when generating images of a hip prosthesis. The image data representing the trial image is either already in digital form as output from the x-ray imaging device 3, or it is converted into digital form by known means. In the present embodiment it is assumed that the table 1, upon which the patient lies, is a flat-panel detector providing digital x-ray image data. The trial image is, in effect, a two-dimensional (2D) representation of the imaged region of the patient's body.

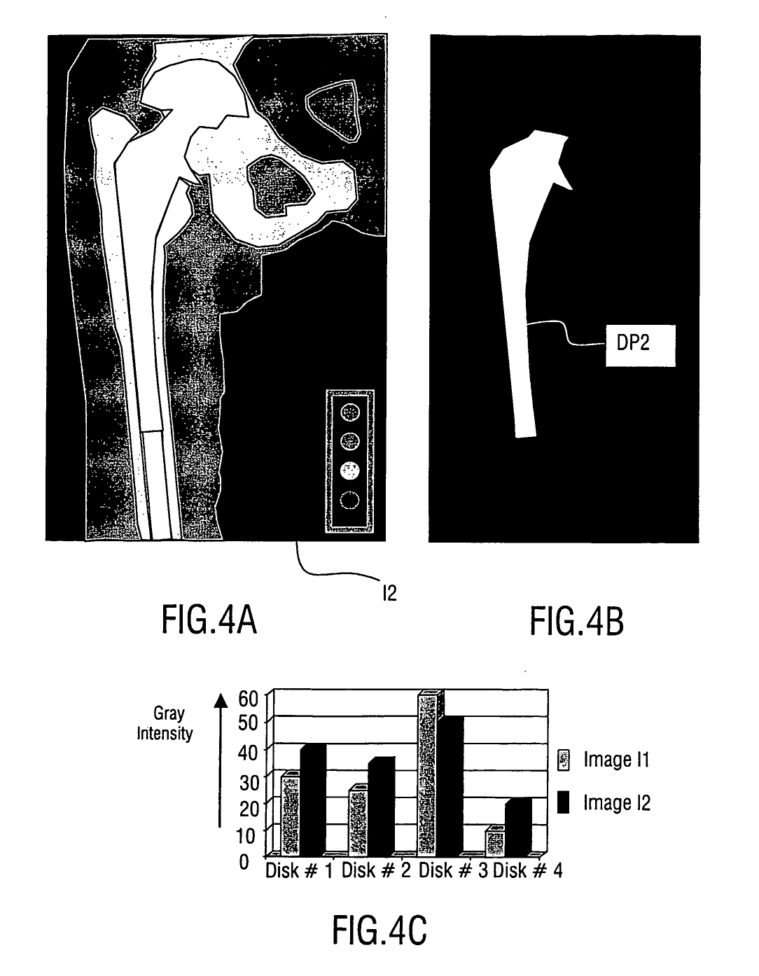

FIG. 4A shows a schematic drawing for representing an example of a typical x-ray trial image that would be obtained of a hip prosthesis. The hip prosthesis is represented by the brightest portion of the image. The hip prosthesis comprises a lower portion fixed in the femur (long bone), which portion forms a joint with an upper portion fixed in the hip. The joint between the lower and upper portions is not visible since the upper portion is positioned in rotation around a head formed at the upper part of the lower portion and since the image of FIG. 4A is a 2D projection view. The drawing of FIG. 4A also shows parts having different gray levels representing the bones and the soft tissues of the patient.

Next, the system has processing means to perform a processing step S2 applied to the digital trial image data of FIG. 4A, using known techniques, to identify the outline of the feature of interest. So, for a given 2D x-ray trial image 12, the outline of the prosthesis is determined in step S2. In fact, for a hip prosthesis, only a portion called discriminating part DP2 of the outline is needed, and is always visible. Such a discriminating portion DP2 is for example the part of the lower portion of the hip prosthesis that is fixed in the femur, represented as the bright element shown in FIG. 4B. Thus, for such a case, the outline of the discriminating portion DP2 is identified in the processing step S2.

As shown in FIG. 2, the viewing system comprises means to perform a preliminary acquisition step S0 of acquiring a 3D representation of the same feature of interest. The reference 3D representation can be acquired in a number of ways. For example, digital data representing a 3D computer-aided design (CAD) model of the prosthesis may be available from the manufacturer thereof. Another possibility for acquiring a reference 3D representation is to perform 3D imaging of the hip region of the patient straight after the operation implanting the hip prosthesis. This imaging process would cover the prosthesis and a region around it.

Next, in an example, the viewing system has means to perform a step S3, wherein the pose of the hip prosthesis in the trial image is estimated by comparing the digital trial image data with a reference 3D representation of the same anatomical feature, as acquired in step S0. The pose of the prosthesis in the trial image is estimated by determining which 2D projection of the reference 3D representation matches most closely with the outline of the prosthesis in the trial image.

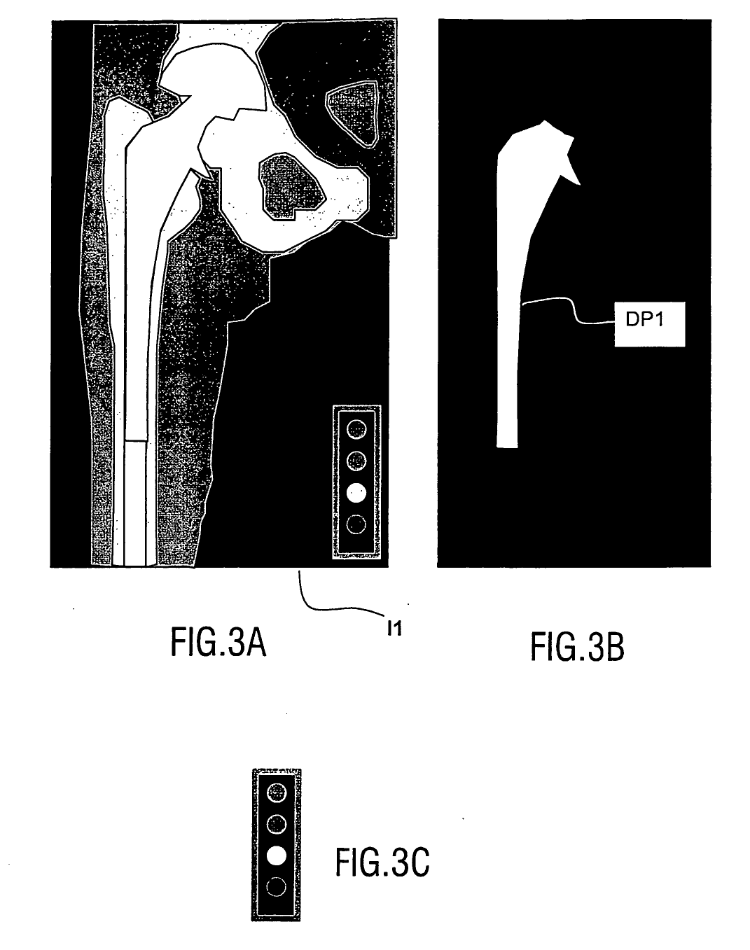

A 2D projection, called reference image I1, determined from the reference 3D representation, is shown as an example in FIG. 3A. The corresponding discriminating part, denoted by DP1, of the outline of the feature of interest is shown in FIG. 3B. In FIG. 3A and FIG. 3B, the hip prosthesis and the discriminating part are the brightest parts.

The outline of the discriminating part DP2 as shown in FIG. 4B, is compared with that of the computer simulated 2D projection DP1 of the 3D model of the prosthesis. The pose, i.e. viewing angles and geometrical parameters of the true prosthesis are determined from the results of this comparison, as the simulated projection geometry that results in the best possible agreement between projected model and true prosthesis outlines.

The procedure that is used to estimate the pose of the outline of the prosthesis in the trial image of FIG. 4B, with respect to the reference 3D representation, is preferably the pattern-matching process described in the article by Sarojak et al cited above. This estimation procedure yields the prosthesis pose in terms of the angle of the discriminating part of the prosthesis relative to the 2D image frame co-ordinate system.

Now, when a desirable pose has been determined in step S3, it is proposed as input requirement. The system has means to automatically calculate in a step S4, the geometrical parameters forming the settings of the x-ray apparatus in use, and particularly of the imaging device 3 for reproducing the required pose. Thus, the calculation means of the imaging system performs the step S4 to transform the estimated pose data so as to produce imaging means control data indicating one or more settings of the imaging device 3 required in order to produce a corrected image, called final image, in which the prosthesis has a desired pose.

According to the above-cited examples, the desired pose may be the reference pose selected by the medical practitioner, for example from a consideration of the 3D reference model. Alternatively, the desired pose may be derived from the preliminary image of the prosthesis, for example, the previously generated 3D image of the prosthesis. As illustrated by FIG. 1C, in general, the desired pose will be specified in terms of the desired coordinates X, Y, and of the desired angle Ψ of the prosthesis with respect to the 2D image frame co-ordinate system.

This desired angle is compared with the estimated pose data and control data are produced indicating how the position of the imaging co-ordinate frame relative to the patient must be set or changed in order that a subsequent image produced by the imaging apparatus will present the prosthesis in the desired pose.

The system has further means to perform a step S5 of providing this information to the user, for the user to be able to take a further image called final follow-up image, with closer viewing parameters with respect to the reference. In this embodiment, the calculated control data is then displayed on the display screen 6, at step S5, and constitutes instructions to the operator as to how to set up the geometry of the imaging apparatus in order to produce a further image, called final image, in which the prosthesis has the desired pose.

As, illustrated by FIG. 1B, the geometrical parameters that are estimated as settings for the examination apparatus will be used to adjust the geometrical parameters constituted by the coordinates of the imaging device 3 determined along the three axes x, y, z of an orthogonal referential; by the angles θ, φ, Ψ of the imaging device 3 determined in the three orthogonal plans of said referential with respect to said axes; and by the distance between the imaging device and the patient or the bed, which defines the magnification of the images. So, the pose is said to be substantially the same when the seven geometrical parameters above-cited are the same or substantially near while viewing the prosthesis in the image of reference and viewing the prosthesis in the final follow-up image.

Typically, as above-described in reference to FIG. 1A and 1B, the geometrical parameters of the x-ray imaging device 3 relative to the bed 1 can be changed by changing the translation position of the imaging device in x, y and z (three degrees of freedom), by altering the angle of the head (azimuth angle Ψ, elevation angle φ, and/or in-plane rotation angle θ, forming three more degrees of freedom), by changing the distance of the imaging device that changes the imaging magnification ratio. Some of these possibilities are indicated by the arrows shown in FIG. 1A.

The control data output from the data processing means in step 5 indicates how one or more of these parameters should be set or changed. The control data could indicate, for example, that the elevation angle of the imaging head should be set at −80°, or increased by 5°, etc.

In practice, once the operator has set-up the x-ray medical examination apparatus, in a further step S6, according to the control data then the final image can be generated or, alternatively, another trial image can be generated and displayed in step 7. The pose of the prosthesis therein can be estimated in order to check whether the desired pose has been achieved. If the desired pose has not yet been achieved, then further control data can be output and displayed to the operator. Thus, the process can be repeated so that the imaging angles approach more closely to the “reference”.

In the embodiment operated as described above, a patient is presented to the x-ray medical examination apparatus, an initial trial image is produced of a region including the anatomical feature of interest, the pose of that anatomical feature is estimated by comparison with a reference model and the estimated pose data is transformed so as to produce medical examination apparatus control data to alter the geometry of the x-ray imaging device relative to the patient and produce a final image having the anatomical feature in a desired pose.

However, it is also possible to treat as the “trial” image an earlier image of the anatomical feature in question, produced at an earlier session with the same patient. In such a case the pose of the prosthesis in the earlier image is estimated, and the medical examination apparatus control data is controlled, for example, so as to set-up the x-ray imaging device to produce an image having the feature of interest in the same pose as in the earlier (trial) image.

It is advantageous to be able to control the patient's position relative to the x-ray medical examination apparatus so as to eliminate positioning errors due to the patient lying in different positions when the trial image and the final image are generated, respectively. This is not such an issue when the trial image and final image are generated at different times during a single session, but is more significant when these images are generated at different sessions. Control of the patient's position relative to the medical examination apparatus can be achieved in numerous different ways, for example by providing on the bed (or other emplacement for the patient) alignment marks to be lined up with, for example, the patient's feet, head and pelvis, by measuring distances from points on the bed to specific body parts, by providing brackets which physically position the patient relative to the x-ray imaging apparatus, etc.

Comparison of medical images is rendered difficult for the medical practitioner not only because of differences in imaging geometry in the compared images but also due to differences in image intensity characteristics of the different images. Such differences in image intensity characteristics can arise because physically different apparatuses are used to generate the different images (apparatus of different manufacturers, different machines of a same manufacturer, etc.), or because the same x-ray medical examination apparatus is set-up differently when the various images are generated. Accordingly, it is advantageous for the viewing system of the present invention to be able to produce medical examination apparatus control data relating to settings of the x-ray medical examination apparatus, which affect the intensity (gray level) characteristics of the image, as well as relating to the imaging geometry.

Often, no data is recorded regarding what were the settings of the x-ray medical examination apparatus at the time when an image was generated. Thus, in order to generate control data relating to intensity settings of the x-ray medical examination apparatus, it is preferred that the image data processing means of the above-described first embodiment can implement additional processing on the trial image to assess the image intensity profile of the apparatus used to produce this trial image. The assessed intensity profile data is transformed to produce control data relating to intensity settings of the x-ray medical examination apparatus. Typically, the control data will indicate how one or more of the following parameters should be set or changed: voltage of the exposition light generator and/current intensity of said exposition light generator, which may be the x-ray source generator.

For generating control data relative to the setting of the exposition source, a calibration phantom is added to the image field so as to fully characterize the x-ray generator setting. A computer program then characterizes the “apparent” x-ray setting from the gray intensities of the phantom pattern. In this disposition, standard image analysis techniques allow to locate the different sub-parts of the calibration phantom and record in a table the gray levels of the set of representative sub-parts of the calibration phantom, which have known x-ray absorption properties. This procedure is applied to the reference image, which will later be compared with trial and follow-up images. This allows of relating the gray appearance of the image to the x-ray absorption properties of the imaged objects.

As illustrated in FIG. 3A, a series of filters for forming a calibration phantom is positioned near the patient while taking the view for forming the reference image. As illustrated by FIG. 4A, a corresponding series of filters for forming a calibration phantom is positioned near the patient while taking the view for forming the trial and then the follow-up image. The calibration phantoms have corresponding sub-parts, each sub-part filtering differently the exposition x-ray irradiation, as illustrated in FIG. 3C.

This operation of calibrating the gray levels is useful for images that are originally generated in digital form or converted from an analogue form by known means. This can also help to compensate for aging related changes in the imaging systems sensitivity.

As illustrated in FIG. 4A, in the trial or follow up image, the corresponding calibration phantom is examined and the gray intensities of its sub-parts are again recorded in another tabulation. If the relative gray values of bright and dark sub-parts in the follow-up image are closer to each other than in the initial reference image, the practitioner is advised to reduce the accelerating voltage of his x-ray generator and vice versa if this ratio comparison is the other way around. FIG. 4C illustrates the corresponding gray intensities in reference image P1 and in trial or follow-up image P2. On the other hand, the gray dynamic range increases with the x-ray generator current. This is illustrated by the larger difference between maximum and minimum gray intensities in image P2 as compared to those differences in image P1 in FIG. 4C. Then, the doctor is provided with indications to increase or decrease the x-ray generator current when necessary, while avoiding saturation of the imaging system. The indications of voltage settings and current settings are displayed on the screen 6.

The above described dispositions comprising geometrical settings and exposition light settings should allow the medical practitioner to reproduce very similar x-ray generator parameters for the reference and follow-up images for display in step S7.

In the above-described embodiment, the medical examination apparatus control data are output in a viewable form and constitutes instructions to the operator as to how to set-up the imaging apparatus relative to the patient. However, in an other embodiment of the present invention, the medical examination apparatus control data are output directly to said x-ray medical examination apparatus and automatically control the geometry thereof and/or image intensity profile applied thereby. The x-ray medical examination apparatus must, of course, allow this automatic control to be performed. The necessary capability can be built into the medical examination apparatus at the time of manufacture, or retro-fitted.

The drawings and their description herein-before illustrate rather than limit the invention. It will be evident that there are numerous alternatives that fall within the scope of the appended claims. In this respect the following closing remarks are made.

As mentioned above, the imaging apparatus is not limited to x-ray devices and the imaged feature can be substantially any anatomical feature including artificial elements such as prostheses/implants. Furthermore, in the above-described embodiments of the invention, the pose of an anatomical feature in an image is estimated using a pattern-matching technique with reference to 2D projections from a 3D reference, but other pose estimation techniques can be used.

Any reference sign in a claim should not be construed as limiting the claim.

1. Medical examination apparatus comprising imaging means, a viewing system and image data processing means, wherein the image data processing means comprises;
pose estimation means for processing data relating to an image generated by the imaging means so as to estimate data representing the pose of an anatomical feature appearing in the image;
pose correction means for processing data, generated by the pose estimation means, representing the pose of the anatomical feature so as to produce imaging means control data for controlling one or more parameters of the imaging means indicative of the desired imaging conditions for producing a further image having the anatomical feature in the pose estimated by the pose estimation means, and
data output means for outputting said imaging means control data.

2. The apparatus according to claim 1, wherein, in use, the data output means outputs the imaging means control data in a viewable form.

3. The apparatus according to claim 1, wherein, in use, the data output means outputs the imaging means control data to the imaging means.

4. The apparatus according to claim 1, wherein the imaging means control data further comprises data to control one or more parameters of the imaging means for producing a further image having desired intensity characteristics.

5. The apparatus according to claim 1, wherein, in use:
the pose estimation means processes data relating to a first and a second images, respectively generated by the imaging means at different times, so as to estimate the relative pose of an imaged anatomical feature in the second image compared with the pose thereof in the first image, and
the pose correction means processes data generated by the pose estimation means representing the relative pose of the anatomical feature so as to produce imaging means control data indicative of settings of the imaging means required to produce a further image having the anatomical feature in the same pose as the pose thereof in the first image.

6. A medical viewing system comprising:
pose estimation means for processing data relating to an image generated by an imaging means so as to estimate the pose of an anatomical feature appearing in the image,
pose correction means for processing data, generated by the pose estimation means, representing the pose of the anatomical feature so as to produce imaging means control data indicative of the desired imaging geometry for controlling one or more parameters of the imaging means for producing a further image having the anatomical feature in the pose estimated by the pose estimation means, and
data output means for outputting said imaging means control data.

7. The medical viewing system according to claim 6, wherein, in use, the data output means outputs the imaging means control data in a viewable form.

8. The medical viewing system according to claim 6, wherein, in use, the data output means outputs the imaging means control data to the imaging means.

9. The medical viewing system according to claim 6, wherein the imaging means control data further comprises data to control one or more parameters of the imaging means whereby to produce a further image having desired intensity characteristics.

10. The medical viewing system according to claim 6, wherein, in use:
the pose estimation means processes data relating to a first and a second images, respectively generated by the imaging means at different times, so as to estimate the relative pose of an imaged anatomical feature in the second image compared with the pose thereof in the first image, and
the pose correction means processes data generated by the pose estimation means representing the relative pose of the anatomical feature so as to produce imaging means control data indicative of settings of the imaging means required to produce a further image having the anatomical feature in the same pose as the pose thereof in the first image.

11. A computer program product adapted, when in use on a general-purpose computer, to cause the computer to perform the following steps:
to process data relating to an image generated by an imaging means so as to estimate the pose of an anatomical feature appearing in the image,
to process data, resulting from the pose estimation step, representing the pose of the anatomical feature so as to produce imaging means control data indicative of the desired imaging geometry for controlling one or more parameters of the imaging means for producing a further image having the anatomical feature in the pose estimated by the pose estimation means, and
to output said imaging means control data.

12. Computer program product according to claim 11, wherein the data outputting step comprises outputting the imaging means control data in a viewable form.

13. Computer program product according to claim 11, wherein the data outputting step comprises outputting imaging means control data to the imaging means.

14. Computer program product according to claim 11, wherein the imaging means control data further comprises data to control one or more parameters of the imaging means for producing a further image having desired intensity characteristics.

15. Computer program product according to claim 11, wherein:
the pose estimating step comprises processing data relating to a first and a second images, respectively generated by the imaging means at different times, so as to estimate the relative pose of an imaged anatomical feature in the second image compared with the pose thereof in the first image, and
the pose correcting step comprises processing data, resulting from the pose estimation step, representing the relative pose of the anatomical feature so as to produce imaging means control data indicative of settings of the imaging means required to produce a further image having the anatomical feature in the same pose as the pose thereof in the first image.

16. The apparatus according to claim 2, wherein the imaging means control data further comprises data to control one or more parameters of the imaging means for producing a further image having desired intensity characteristics.

17. The apparatus according to claim 1, wherein the imaging means control data further comprises data to control one or more parameters of the imaging means for producing a further image having desired intensity characteristics.

18. The apparatus according to claim 2, wherein, in use:
the pose estimation means processes data relating to a first and a second images, respectively generated by the imaging means at different times, so as to estimate the relative pose of an imaged anatomical feature in the second image compared with the pose thereof in the first image, and
the pose correction means processes data generated by the pose estimation means representing the relative pose of the anatomical feature so as to produce imaging means control data indicative of settings of the imaging means required to produce a further image having the anatomical feature in the same pose as the pose thereof in the first image.

19. The apparatus according to claim 3, wherein, in use:
the pose estimation means processes data relating to a first and a second images, respectively generated by the imaging means at different times, so as to estimate the relative pose of an imaged anatomical feature in the second image compared with the pose thereof in the first image, and
the pose correction means processes data generated by the pose estimation means representing the relative pose of the anatomical feature so as to produce imaging means control data indicative of settings of the imaging means required to produce a further image having the anatomical feature in the same pose as the pose thereof in the first image.

20. The apparatus according to claim 4, wherein, in use:
the pose estimation means processes data relating to a first and a second images, respectively generated by the imaging means at different times, so as to estimate the relative pose of an imaged anatomical feature in the second image compared with the pose thereof in the first image, and
the pose correction means processes data generated by the pose estimation means representing the relative pose of the anatomical feature so as to produce imaging means control data indicative of settings of the imaging means required to produce a further image having the anatomical feature in the same pose as the pose thereof in the first image.
